(19) United States
(12) Patent Application Publication
(10) Pub. No.: US 2024/0252524 A1
(43) Pub. Date: Aug. 1, 2024

(54) Process to Reduce Ivermectin Particle Size

(71) Applicant: Hovione Scientia Limited, Cork (IE)

(72) Inventors: Luis SOBRAL, Montijo (PT); Luis Gaspar SOUSA, Amadora (PT); Sara SEQUEIRA, Sintra (PT)

(21) Appl. No.: 18/565,724

(22) PCT Filed: May 31, 2022
(86) PCT No.: PCT/GB2022/051381

§ 371 Date: Nov. 30, 2023

(30) Foreign Application Priority Data
Jun. 1, 2021 (PT) 117268

(51) Int. Cl. A61K 31/7048; A61K 9/14

(52) U.S. Cl. CPC A61K 31/7048; A61K 9/145

(57) ABSTRACT

The invention provides a process to reduce the particle size of ivermectin wherein the process comprises incorporating ivermectin particles in a wet or liquid medium and subjecting the medium comprising the particles to mixing in a high shear mixer.

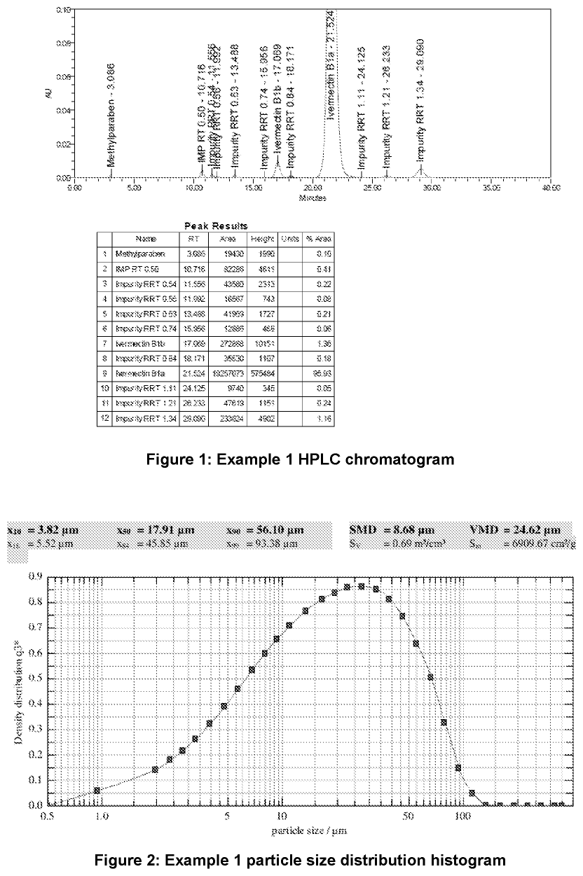

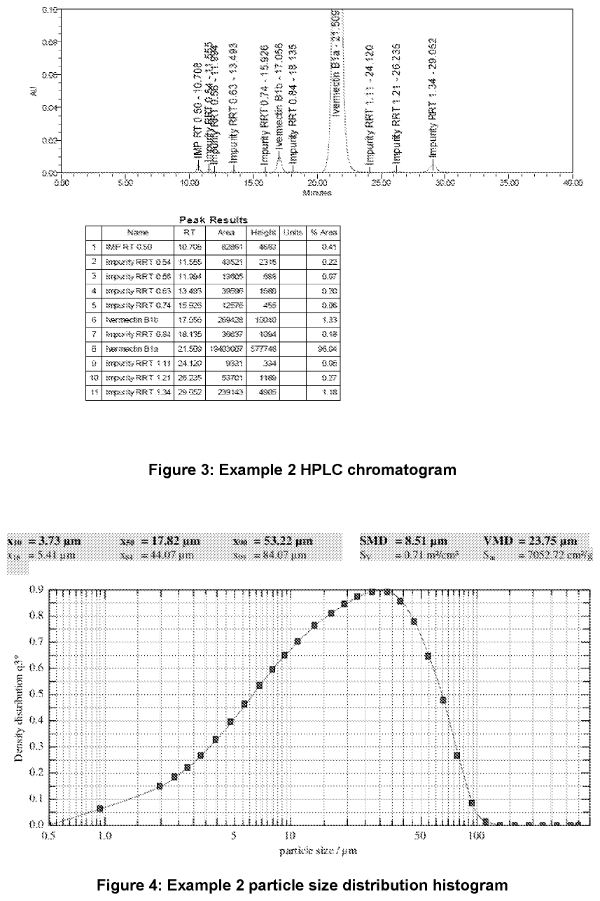

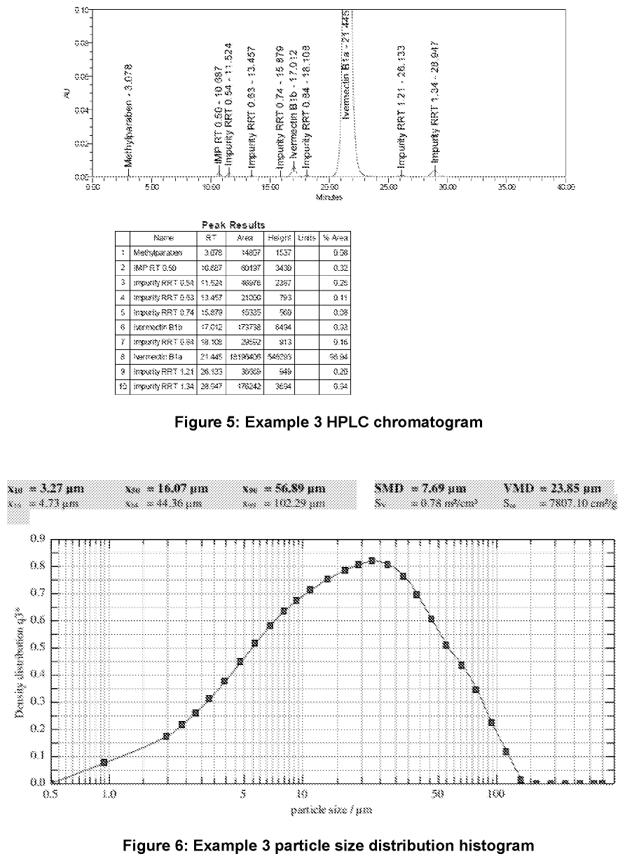

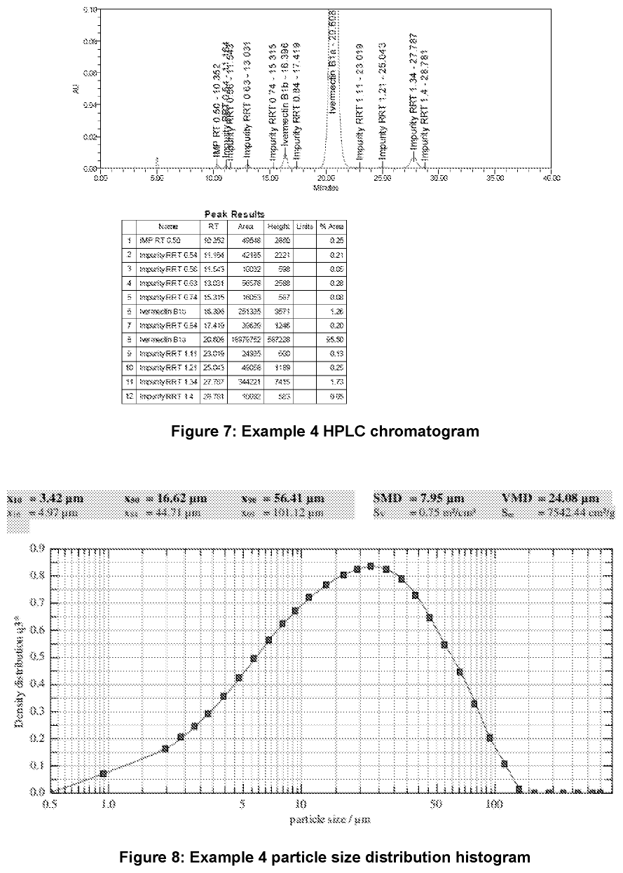

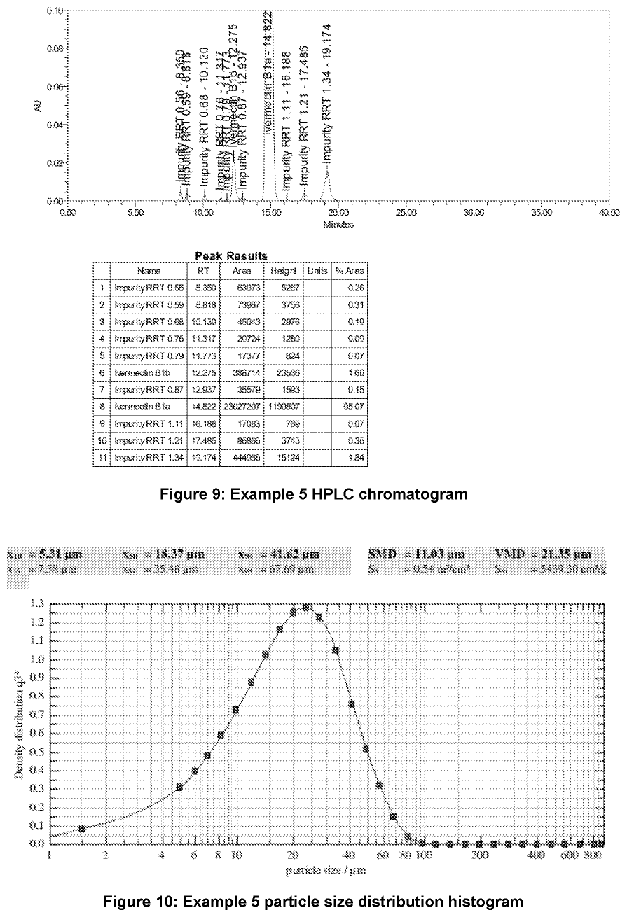

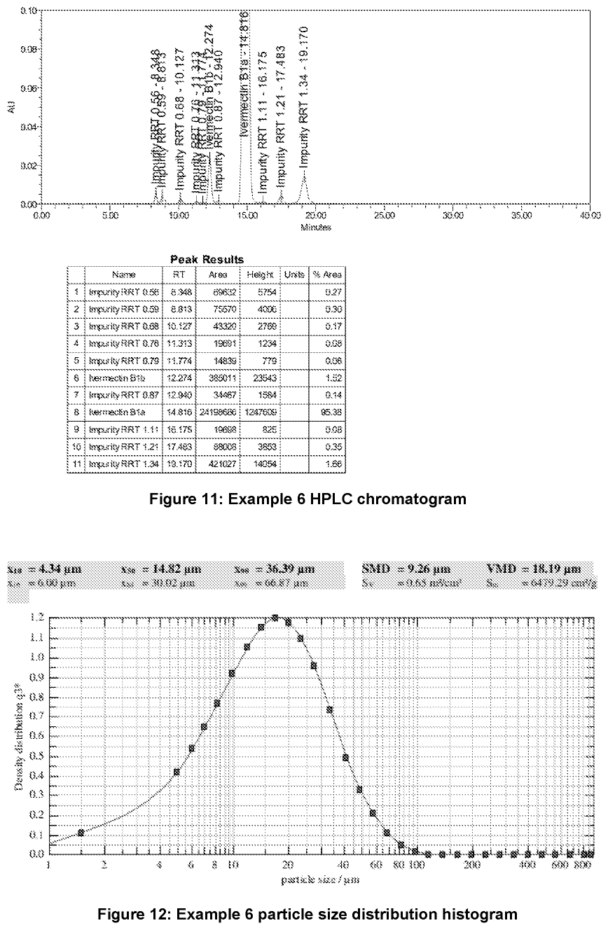

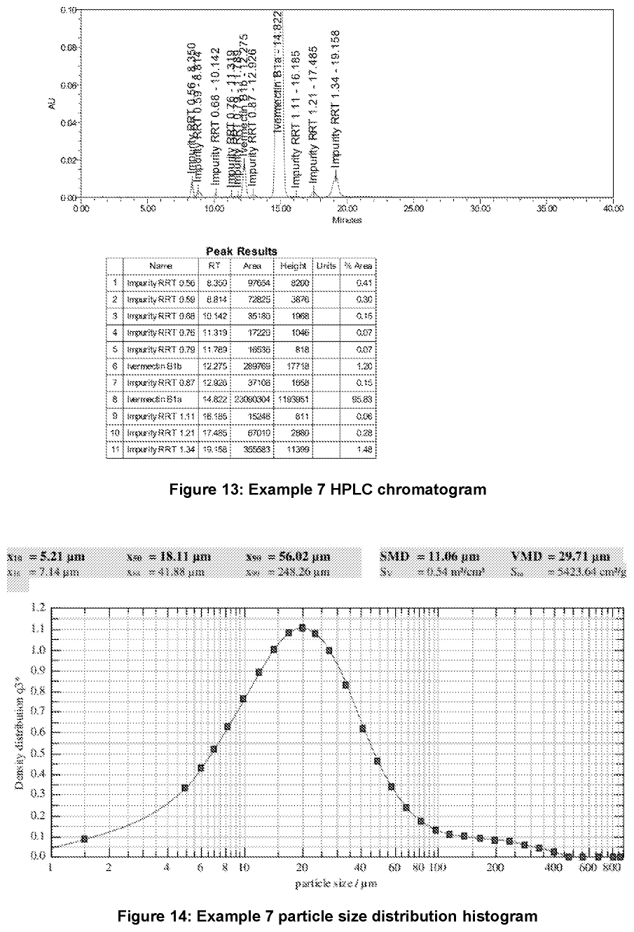

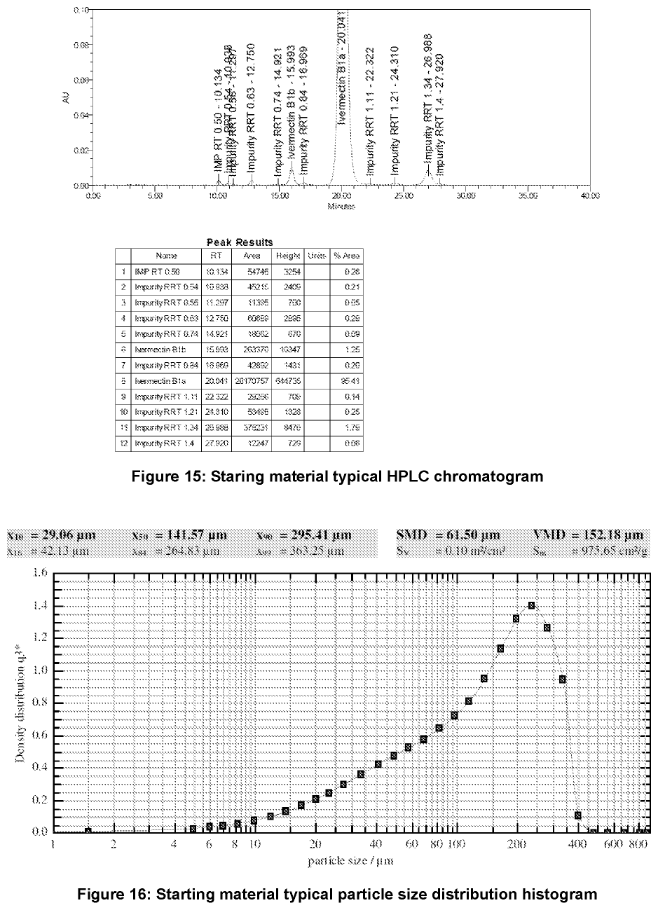

### FIELD OF THE INVENTION

The present invention relates to a new process designed to control the particle size and the related substances content of ivermectin. In one aspect, the process comprises the micronization of ivermectin in a wet or liquid medium, to reduce the particle size to levels adequate for downstream formulation. In a preferred aspect, the process also comprises the purification of ivermectin in the solvent mixture used to micronize the product. The purification of ivermectin occurs by purge of related substances during the micronization. Another important feature of the process is that it comprises preventing the decomposition of ivermectin by air oxidation during the micronization, using adequate antioxidants acting as radical scavengers. The antioxidants also prevent the air oxidation of ivermectin during storage.

### BACKGROUND OF THE INVENTION

Ivermectin, compound of molecular structure (I), is an anti-helminthic active pharmaceutical substance prescribed for the treatment head lice, scabies, river blindness, strongyloidiasis and lymphatic filariasis, among other diseases promoted by parasites.

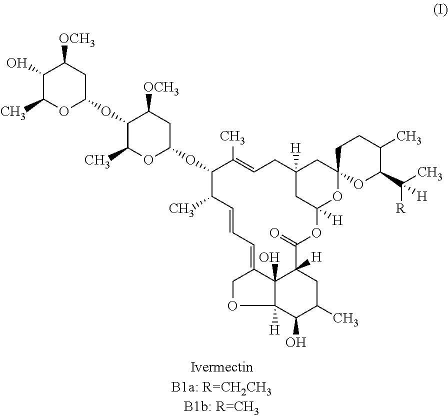

It is constituted by 80% or more of the component B1a and 20% or less of the component B1b. Compound I was first disclosed in U.S. Pat. No. 4,199,569, wherein a method for its preparation was described comprising the hydrogenation of abamectin, compound II, constituted by 80% or more of avermectin B1a and 20% or less of avermectin B1b, in the presence of the catalyst tris(triphenylphosphine) rhodium (I) chloride, the Wilkinson catalyst.

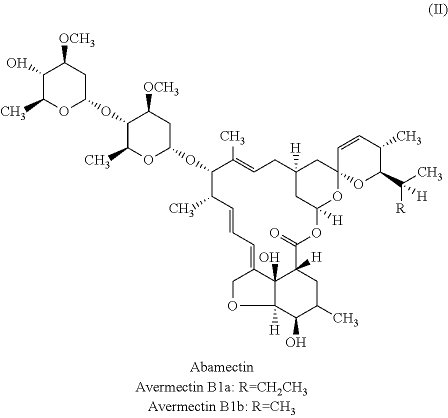

The process describes two consecutive recrystallizations to isolate ivermectin after the hydrogenation. The first recrystallization is carried out in a mixture of ethanol, formamide and water (4:10:2), wherein ivermectin is dissolved at 40-50° C. and afterwards is crystallized by slow cooling, under overnight stirring. The second recrystallization is carried out in a mixture of ethanol and water (4:4), by dissolving at 35-40° C. the product obtained from the first crystallization, followed by slow cooling under overnight stirring. Under the conditions described by the inventors, ivermectin is obtained from abamectin in 83% weight yield. The authors claim ivermectin in a mixture which contains about 80% of the component B1a and 20% of the component B1b

In a publication describing the analytical profile of ivermectin (Analytical Profiles of Drug Substances, vol. 17, 1988, pages 155 to 184), the author described stability characteristics of the product in solution and in the solid state. According to the author, ivermectin contains many functional groups and it is instable in acidic and basic solutions. The rate of degradation of the product increases with both increased acidity and increased basicity. In acidic solution ivermectin suffers hydrolyses reactions of the two sugar rings to yield the monosaccharide and the aglycone by-products. In basic solution ivermectin undergoes isomerization to yield the 2-epi-ivermectin and the Δ²-ivermectin by-products. It is noted that the optimum pH for ivermectin stability in solution is 6.3. The predominant mode of degradation of ivermectin under neutral conditions is the oxidation which generates by-products at the 8a position, namely, the 8a-oxo ivermectin and the 8a-hydroperoxide ivermectin. Ivermectin is also photolabile and the products resulting from its photo degradation are geometric isomers at the C8-C9 and C10-C11 olefins. The author also reports that, neat ivermectin, in the absence of extraneous reactants and impurities, it is a stable molecule in its crystalline state. Nonetheless, the air oxidation of ivermectin in the solid-state is a known phenomenon which is recognized by the fact that the impurity 8a-oxo ivermectin is listed in ivermectin related substances test of both the European and the United States pharmacopoeias.

The U.S. Pat. No. 6,072,052 claims a process to prepare ivermectin by selective hydrogenation of abamectin with rhodium salts and rhodium-phosphine complexes containing hydrazine. The inventors describe a process where the ivermectin obtained from the hydrogenation is purified by removal of the catalyst by extraction with lipophilic solvents, which dissolve well the catalyst but do not dissolve significantly the ivermectin. The lipophilic solvents such as aliphatic hydrocarbons are added to the mixture, to dissolve the catalyst and, according to the inventors, the ivermectin precipitates out of the mixture. The inventors refer obtaining crude ivermectin which contains 1.3% of avermectin, 94.8% of ivermectin and 25% of tetrahydro avermectin by HPLC.

U.S. Pat. No. 6,265,571 claims a process to purify ivermectin by reverse phase flash column chromatography, wherein the chromatographic column comprises a C18 silica gel column and the eluent comprises a mixture of acetonitrile with a lower alkyl alcohol and water. The inventors report obtaining ivermectin with 95.15% of the component B1a and 2.22% of the component B1b, by HPLC. The inventors claim a process wherein the purity level of the purified ivermectin comprises at least about 98%. The inventors report that the purity of ivermectin is calculated by adding to the area % of the component B1a the area % of the component B1b and deducting the residual contents of water and volatiles. The implementation of column chromatography in the pharmaceutical industry presents several disadvantageous related to the requirement of large pumps operating at high pressures, large consumptions of solvents leading to large volumes of liquid waste and long process times.

WO 2019/180417 A1 claims amorphous ivermectin which is obtained by spray drying a solution of ivermectin prepared by dissolving ivermectin in an organic solvent or mixtures of organic solvent or mixtures of an organic solvent with water. The inventors refer ethanol, methyl ethyl ketone, acetone or 1-butanol as solvents which can be used to prepare the solution of ivermectin. The inventors claim amorphous ivermectin with particle size distribution between 0.1 μm and 20 μm. No data on the purity of ivermectin obtained according to the process claimed is disclosed.

Ivermectin has been used to treat various diseases caused by ectoparasites and endoparasites, in humans and other animals. It is commercially available in pharmaceutical forms of oral tablets (Stromectol®), oral suspension (compounding preparation e.g. Wedgewood Pharmacy's), chewable tablets (Tri-Heart Plus®), lotion for topical aministration (Skilice®) and cream (Rosiver® and Soolantra®). Many other pharmaceutical forms have been under development and these include sub-cutaneous formulations (Sharun K, Shyamkumar T S, Aneesha V A, Dhama K, Pawde A M, Pal A (2019), Current therapeutic applications and pharmacokinetic modulations of ivermectin, Veterinary World, 12(8): 1204-1211). Ivermectin is a compound with low solubility in water and high permeability, being classified as a BCS class II drug. Its low water solubility and poor stability in aqueous preparations present significant challenges on the manufacture of formulations. The manufacture of pharmaceutical formulations requires several physicochemical features from the active pharmaceutical substance in order to ensure consistent bioavailability. The prior art reports modification of ivermectin pharmacokinetic properties by altering the type of formulation (Albert Lo, P K., Fink, D. W., Williams, J. B. et al. Pharmacokinetic studies of ivermectin: Effects of formulation. Vet Res Commun 9, 251-268 (1985)). Additionally, a requirement for several types of formulations is that the active pharmaceutical substance presents reduced particle sizes, to ensure homogeneous distribution of the active substance over the pharmaceutical form.

Heretofore, the process of reducing the particle size of solid ivermectin particles has been carried out as a dry process, typically using jet milling. We have now appreciated that there is an unmet need in the prior art related to processes with a capacity to reduce the particle size of ivermectin and simultaneously increase its purity which provides advantages for formulation processes. The process of this invention is able to reduce the particle size of ivermectin in a wet medium, through a high shear mixer technique, using solvent mixtures which enable purge of impurities, generating purer ivermectin. Another relevant aspect of this process is that it comprises the use of antioxidants which prevents the air oxidation of ivermectin during processing and also during storage. High shear mixer processes generate strong shear forces and high temperatures which may promote degradation of the products when submitted to such conditions. Accordingly, such processes have never been considered suitable for use with ivermectin. Unexpectedly, the present inventors have now found that the process of this invention does not promote the decomposition of ivermectin which is a sensitive compound prone to suffer several side reactions.

### SUMMARY OF THE INVENTION

According to the present invention, there is provided a process to reduce the particle size of ivermectin wherein the process comprises incorporating ivermectin particles in a wet or liquid medium and subjecting the medium comprising the particles to mixing in a high shear mixer.

High shear mixing as a technique is well understood in the art. In the invention, any suitable high shear mixer equipment may be used, including a batch or in-line high shear mixer, or an ultra-high-shear mixer. High shear mixing, as opposed to low shear mixing, can suitably be understood for the purposes of the present invention as a mixing process which has the ability to reduce the average size of solid particles of the API material. Low shear mixing does not, in general, reduce the average particle size of an API.

The present invention thus discloses a process to reduce the particle size of ivermectin in a wet or liquid medium comprising, in one preferred aspect, an organic solvent and/or mixtures of organic solvent with water and, preferably, formamide.

The present invention discloses a process to reduce the particle size and simultaneously purify ivermectin, thus reducing the total content of related substances in ivermectin. This is a particular, and surprising, advantage of the process, given the sensitive nature of ivermectin. The organic solvent can for example be an alcohol, an ester, a ketone, an ether, an amide, a hydrocarbon or a halogenated hydrocarbon. In a preferred embodiment, the organic solvent is an alcohol and in a more preferred embodiment, the organic solvent is ethanol.

### DETAILED DESCRIPTION OF THE INVENTION

In a preferred aspect, the medium comprising the particles comprises a suspension of ivermectin. Thus, typically, solid particles of ivermectin are suspended in a liquid medium. A liquid medium as used herein means a medium that is in the liquid state at room temperature (e.g. 20-25° C.). The liquid medium suitably comprises an organic solvent or a mixture of an organic solvent with water. The initial particle size of the ivermectin particles may vary but will typically be of the order of a Dv (90) of 200-300 microns or more, as typically results from standard manufacturing processes.

The organic solvent may be any suitable solvent, but preferably comprises, or consists of, an alcohol, an ester, a ketone, an ether, an amide, a hydrocarbon or a halogenated hydrocarbon. One or more of these solvents may be employed, for example as a mixture.

Where the organic solvent is an alcohol, preferably this is an aliphatic alcohol, for example an aliphatic alcohol which is a C₂-C₈ aliphatic alcohol. Especially preferred are ethanol or isopropanol.

Where the organic solvent is an ester, a compound of general formula RCOOR′, preferably this is an ester where R and R′ are alkyl groups, for example, C1-C3 alkyl groups, preferably, ethyl acetate or isopropyl acetate.

Where the organic solvent is a ketone, a compound of general formula RCOR′, preferably this is a ketone where R and R′ are alkyl groups, for example, C1-C₃ alkyl groups, preferably, acetone or methyl ethyl ketone.

Where the organic solvent is an ether, a compound of general formula ROR′, preferably this is an ether where R and R′ are alkyl groups, for example, C₁-C₃ alkyl groups, preferably, diethyl ether or diisopropyl ether. Optionally, the ether is a cyclic ether of general formula RO, for example a C₆ cyclic ether, preferably, tetrahydrofuran, or a cyclic ether of general formula RO₂, for example, a C₄ cyclic ether, preferably dioxane.

Where the organic solvent is an amide, a compound of general formula RR′NCOR″, preferably this is an amide where R and R′ are alkyl groups, for example, C₁ alkyl groups, and R″ is hydrogen or a C₁ alkyl group, preferably, dimethylformamide or dimethylacetamide.

Where the organic solvent is a hydrocarbon, preferably this is an aliphatic hydrocarbon or an aromatic hydrocarbon, for example, a C₆-C₇ aliphatic hydrocarbon, preferably, hexane or heptane, or a C7 aromatic hydrocarbon, preferably toluene.

Where the organic solvent is a halogenated hydrocarbon, preferably this is an aliphatic halogenated hydrocarbon, for example, a C₁ halogenated aliphatic hydrocarbon, preferably, dichloromethane.

The liquid medium comprises an organic solvent, or a mixture of organic solvents, or a mixture of an organic solvent with water, preferably with formamide. The organic solvent is a solvent wherein ivermectin is soluble or freely soluble. Mixtures of an organic solvent with a solvent wherein ivermectin is insoluble, like for example mixtures of an alcohol with water, can be used to carry the high shear mixer process in order to increase the yield of the process. Table 1 summarizes ivermectin solubility data disclosed in the prior art (Analytical Profiles of Drug Substances, vol. 17, 1988, pages 155 to184).

TABLE 1
Solubility data of ivermectin at room
temperature disclosed in the prior art

| | Solubility | Pharmacopoeia |
|---|---|---|
| Solvent | (mg/ml) | Classification |
| Water | 0.000001 | Practically Insoluble |
| n-Hexane | 0.69 | Slightly soluble |
| Diethyl ether | 61 | Soluble |
| Toluene | 63 | Soluble |
| Isopropanol (2-propanol) | 70 | Soluble |
| Acetone | 81 | Soluble |
| Ethanol | 97 | Soluble |
| Methanol | 220 | Freely soluble |
| Ethyl acetate | 240 | Freely soluble |
| Methyl Ethyl ketone | 320 | Freely soluble |
| Tetrahydrofuran | 390 | Freely soluble |
| p-Dioxane | 430 | Freely soluble |
| Dimethylformamide | 510 | Freely soluble |

The proportion of liquid medium to carry out the high shear mixer process can be, for example, from 2 vols. and 30 vols, with respect to ivermectin weight, preferably, from 4 vols. To 8 vols. For mixtures of soluble or freely soluble organic solvents with insoluble solvents, the proportion (by volume) between the soluble solvent and the insoluble solvent can for example be from 10 to 0.01, or from 0.01 and 10, preferably, from 8 to 1.

The proportion, with respect to ivermectin weight, between formamide and ivermectin can be for example from 0.1 vols. to 2 vols., preferably, from 0.2 vols. to 0.4 vols.

The proportion, with respect to ivermectin weight, between the antioxidant and ivermectin can be for example from 0.00001 to 0.001, preferably, from 0.0001 to 0.0003.

The liquid medium thus typically comprises, or consists of, an organic solvent and/or mixtures of an organic solvent with water. In a preferred aspect, the process comprises micronisation by high shear mixing of ivermectin particles in a mixture of an aliphatic alcohol, for example an aliphatic alcohol which is a C2-C8 aliphatic alcohol and water. A mixture of an aliphatic alcohol which is a C₂-C₄ aliphatic alcohol and water may, for example, be used. Particularly preferred is a mixture of ethanol and water.

In a preferred aspect of the invention, an antioxidant may be added to the solvent or solvent mixture (i.e. the liquid medium), to prevent air oxidation of ivermectin during recirculation in the high shear mixer.

Thus, in one aspect, the liquid medium comprising the ivermectin particles also comprises an antioxidant. In one example, the antioxidant comprises a paraben derivative, a phenol derivative or a thiol derivative.

Suitably, the antioxidant may comprise, or consist of, an alkyl paraben, such as methyl or ethyl paraben.

Alternatively, or in addition, the antioxidant may comprise, or consist of, an alkylated phenol derivative, suitably an alkylated hydoxyanisole. Preferred examples include butylated hydroxyanisole (BHA), butylated hydroxy toluene or tocopherol. Alternatively, or in addition, the antioxidant may comprise, or consist of, a thiol derivative such as cysteine.

In a further aspect, a process according to the invention is provided wherein the liquid medium comprising the particles of ivermectin further comprises a stabilizer to minimize desolvation of the ivermectin. This is primarily designed to prevent loss of the solvating solvent from the ivermectin particles via dissolution in the liquid medium, although may also contribute to a purification or purging process which leads a reduction in the total impurity levels.

In an example, the stabilizer comprises an aliphatic monocarboxylic acid amide. Preferably, the stabilizer comprises, or consists of formamide. A lower aliphatic alcohol such as ethanol may also, or in addition to formamide (or the like), function as a stabilizer.

The process of the present invention has, quite surprisingly, been found to reduce the particle size of ivermectin particles without adversely affecting the stability of the ivermectin. Indeed, far from resulting in decomposition, the present process has been found to lead to a reduction in total impurity levels (e.g. decomposition products or side reaction products) whilst still retaining good yields of ivermectin.

In one aspect, the invention provides a process wherein the Dv(90) of the ivermectin particles after high shear mixing is significantly reduced—for example, to less than 100 microns, or less than 80 microns, or preferably to less than 60 microns. Particles with a Dv (90) of less than 50 microns, or even less than 40 microns may be obtained, depending upon the length and intensity of the high shear mixing.

The duration of the mixing step may thus be any suitable time, provided it provides the required reduction in particle size. This may vary depending upon the end pharmaceutical formulation designed to incorporate the ivermectin. The ivermectin particles provided by the present invention are particularly suitable for incorporation into oral pharmaceutical formulations. The present process can be employed to provide ivermectin particles with a Dv90 of down to about 30 microns if desired. Mixing times of between 1 to 4 hours are typically required. A preferred mixing time is from about 2 or 2.5 hours to about 3 or 3.5 hours.

We have found that the present process can simultaneously reduce both the particle size of ivermectin and the total level of impurities present within the particles—e.g. degradation or decomposition products of the ivermectin. In one aspect, the invention provides a process wherein the reduction in total impurities as measured by HPLC (% area) of the resulting ivermectin particles compared to the initial ivermectin particles is 10% or more. Expressed alternatively, the present invention provides a process wherein the total impurities as measured by HPLC (% area) of the resulting ivermectin particles is 3.0% or less, optionally 2.8% or less, or 2.7% or less.

In one particularly preferred aspect, the liquid medium comprising the ivermectin particles comprises water; ethanol or isopropanol (or a mixture of both) as the organic solvent; methylparaben or ethylparaben (or a mixture of both) as antioxidant, and an aliphatic monocarboxylic acid amide, such as for example formamide, as a stabilizer.

As noted above, any suitable high shear mixer may be used to carry out the high shear mixing step. Preferably, the high shear mixer is capable of operating over a wide range of rotations per minute (RPM). In one aspect of the invention, in the high shear mixing step, a suspension of ivermectin is recirculated at a range of rotations per minute of the high shear mixer of from 500 RPM to 9000 RPM.

Thus, in a preferred aspect, a suspension of ivermectin is recirculated at a range of rotations per minute of the high shear mixer of from 1000 RPM to 2000 RPM. We have found this range tends to provide a gradual and controlled reduction of particle size over time, which can be well monitored by suitable sampling of the liquid medium (at various time intervals) as will be understood by those skilled in the art.

In a preferred mode, the high shear mixer process is performed under smooth rotations per minute, preferably, at around 2000 RPM.

The high shear mixing step, or indeed the whole process, may be carried out at room temperature or at a lower temperature. In a preferred mode of the operation, the high shear mixing step may be carried out at a temperature of from 0° C. to 5° C.

Thus, in one aspect, there is a provided a process according to the invention wherein in the high shear mixing step the liquid medium comprising the ivermectin particles is recirculated at a temperature of from 0° C. to 25° C. In a further preferred aspect, a suspension of ivermectin may be recirculated at a temperature of from 0° C. and 5° C.

In a further aspect of the invention, the step of incorporating ivermectin particles in a wet or liquid medium may be carried out at room temperature or at a lower temperature. In a preferred mode of the operation, this step may be carried out at a temperature of from 0° C. to 25° C., or more preferably, at a temperature of from 0° C. to 5° C. This step may for example, comprise the incorporation, or addition, of ivermectin to a liquid medium which comprises water (if present), an organic solvent component, an antioxidant component (if present), and a stabilizer component (if present).

In a further aspect of the invention, the process comprises the step of isolating the resulting ivermectin particles (after the high shear mixing step) from the liquid medium. This may, for example, comprise steps of filtration and one or more washing steps. The isolation step, or any part thereof, such as filtration and washing steps, may be carried out at room temperature or at a lower temperature. In a preferred mode of the operation, the isolation step, or any part thereof, may be carried out at a temperature of from 0° C. to 25° C., or more preferably, at a temperature of from 0° C. to 5° C.

In a further aspect of the invention, the process as described herein may further comprise, after the high shear mixing, the step of washing recovered ivermectin particles with antioxidant. We have found that this helps to protect the product from oxidation after processing—for example, during storage. Using such as washing step allows some antioxidant to be incorporated into the final product. This washing step may be part of the isolation step described above.

For this washing step, the antioxidant may for example be an alkyl paraben, for example methyl or ethyl paraben; or an alkylated phenol derivative, such as an alkylated phenol hydroxyanisole such as butylated hydroxyanisole; butylated hydroxy toluene or tocopherol. The antioxidant used during this washing step may be the same or different to the antioxidant used (if present) in the liquid medium comprising ivermectin particles that was subjected to the high shear mixing.

In one aspect, a washing step with antioxidant may be performed at a temperature of from 0° C. to 25° C., preferably at a temperature of from 0° C. and 5° C.

As a result of the novel process of the invention provided herein, and particularly the effect of the process on impurity levels, the invention therefore also provides novel ivermectin particles obtainable by, or obtained by, a process according to the invention described herein.

In a further aspect, there is also provided the use of high shear mixing to reduce the particle size of ivermectin in a suspension of ivermectin particles in a liquid medium.

The liquid medium may comprise an organic solvent or a mixture of an organic solvent with water. The organic solvent may be any suitable solvent, but preferably comprises, or consists of, an alcohol, an ester, a ketone, an ether, an amide, a hydrocarbon or a halogenated hydrocarbon. One or more of these solvents may be employed, for example as a mixture. Where the organic solvent is an alcohol, preferably this is an aliphatic alcohol, for example an aliphatic alcohol which is a C2-C8 aliphatic alcohol. Especially preferred are ethanol or isopropanol.

The liquid medium thus typically comprises, or consists of, an organic solvent and/or mixtures of an organic solvent with water. In a preferred aspect, the process comprises micronisation by high shear mixing of ivermectin particles in a mixture of an aliphatic alcohol, for example an aliphatic alcohol which is a C2-C8 aliphatic alcohol and water. A mixture of an aliphatic alcohol which is a C₂-C₄ aliphatic alcohol and water may, for example, be used. Particularly preferred is a mixture of ethanol or isopropanol, and water.

In a preferred aspect, there is provided the use of high shear mixing to reduce the particle size of ivermectin in a suspension of ivermectin particles in a liquid medium, wherein the liquid medium comprises water, an organic solvent comprising ethanol or isopropanol, and a stabilizer comprising formamide.

It will be understood by those in the art that the ivermectin produced by the process of the invention may be incorporated into a pharmaceutical formulation, along with suitable pharmaceutically-acceptable excipients as required, in order to provide a medicinal product to treat patients in need thereof.

The invention herein described thus comprises a process to simultaneously reduce the particle size and purify ivermectin. The process preferably includes the charge of ivermectin to a solvent mixture containing an antioxidant, recirculation of the resulting suspension in a high shear mixer until achieving the target particle size, stopping of the recirculation and isolation of the product by filtration and drying. The use of an organic solvent as described herein enables reduction of the particle size of ivermectin but, at the same time, enables the purification of ivermectin, thus reducing the total content of impurities.

Ivermectin is a mixture of two major components, component B1a and component B1b, which also contains several related substances. The related substances can be expressed as total content of impurities. The process herein described is able to reduce the total content of impurities in ivermectin. This effect is enabled by the use of the organic solvent in the solvent mixture used to micronize ivermectin.

Under the conditions set forth in this invention, ivermectin is submitted to high shear forces without suffering decomposition. High shear mixing processes provide stressful conditions which promote decomposition of the compounds submitted to such processes. The stressful conditions involve recirculating mixtures of the compounds with solvents, at very high speed, through mechanical systems which generate unaligned forces, driving to strong collisions between the particles and the mechanical parts of the high shear mixer and between the particles themselves. Such collisions break the particles of the product thus reducing their particle size. A side effect of the mechanical collisions is a significant increase in the temperature during the high shear mixer process. Surprisingly, submitting ivermectin, which is an active pharmaceutical substance very sensitive to hydrolyses side reactions, oxidation side reactions and temperature side reactions, to the high shear mixer process of this invention, did not decompose the product. In fact, ivermectin is purified with good yields under the high shear process conditions set forth in this invention.

The following Examples are presented to aid understanding of the invention but are not intended to, and should not be considered to, limit its scope in any way.

### Examples

Example 1: Ivermectin (25 g) with a D (90) of 301 μm and a total impurities content of 2.92% (HPLC area %) was added to a mixture of ethanol (100 ml), water (12.5 ml), formamide (10 ml) and methylparaben (0.0075 g) previously cooled to 0-5° C. The suspension was recirculated through a high shear mixer at 2000 RPM, for about 3 hours, while maintaining the mixture at temperature between 0° C. and 5° C. The product was filtered, washed with a mixture of ethanol (2.5 ml) and water (22.5 ml) previously cooled to 0-5° C. and after with a mixture of water (25 ml) and methyl paraben (0.0075 g) previously cooled to 0-5° C. The wet product was dried at 60° C. to yield 21.75 g of ivermectin with a D (90) of 56 μm and content of total impurities of 2.61% by HPLC (area

Example 2: Ivermectin (25 g) with a D (90) of 301 μm and a total impurities content of 2.92% (HPLC area %) was added to a mixture of ethanol (120 ml), water (15 ml), formamide (6 ml) and BHA (0.0075 g) previously cooled to 0-5° C. The suspension was recirculated through a high shear mixer at 2000 RPM, for about 3 hours, while maintaining the mixture at temperature between 0° C. and 5° C. The product was filtered, washed with a mixture of ethanol (3 ml) and water (27 ml) previously cooled to 0-5° C. and after with a mixture of water (30 ml) and BHA (0.0075 g) previously cooled to 0-5° C. The wet product was dried at 60° C. to yield 26.1 g of ivermectin with a Dᵥ(90) of 53 μm and content of total impurities of 2.63% by HPLC (area %).

Example 3: Ivermectin (50 g) with a D (90) of 301 μm and a total impurities content of 2.21% (HPLC area %) was added to a mixture of ethanol (100 ml), water (50 ml), formamide (10 ml) and methylparaben (0.0075 g) previously cooled to 0-5° C. The suspension was recirculated through a high shear mixer at 2000 RPM, for about 3 hours, while maintaining the mixture at temperature between 0° C. and 5° C. The product was filtered, washed with a mixture of ethanol (5 ml) and water (55 ml) previously cooled to 0-5° C. and after with a mixture of water (50 ml) and methyl paraben (0.0075 g) previously cooled to 0-5° C. The wet product was dried at 60° C. to yield 45.5 g of ivermectin with a Dᵥ(90) of 57 μm and content of total impurities of 2.06% by HPLC (area %).

Example 4: Ivermectin (30 g) with a D (90) of 295 μm and a total impurities content of 3.33% (HPLC area %) was added to a mixture of water (150 ml), isopropanol (75 ml), formamide (6 ml) and methylparaben (0.0075 g) previously cooled to 0-5° C. The suspension was recirculated through a high shear mixer at 2000 RPM, for about 3 hours, while maintaining the mixture at temperature between 0° C. and 5° C. The product was filtered, washed with a mixture of ethanol (3 ml) and water (27 ml) previously cooled to 0-5° C. and after with a mixture of water (30 ml) and methyl paraben (0.0075 g) previously cooled to 0-5° C. The wet product was dried at 60° C. to yield 29.4 g of ivermectin with a Dᵥ(90) of 56 μm and content of total impurities of 3.03% by HPLC (area %).

Example 5: Ivermectin (4 g) with a Dᵥ(90) of 300 μm and a total impurities content of 3.22% (HPLC area %) was added to a mixture of water (16 ml), ethanol (16 ml) and formamide (1.6 ml) previously cooled to 0-5° C. The suspension was recirculated through a high shear mixer at 1000 RPM, for about 2 hours, while maintaining the mixture at temperature between 0° C. and 5° C. The product was filtered, and the wet product was dried at 60° C. to yield 3.66 g of ivermectin with a Dᵥ(90) of 42 μm and content of total impurities of 2.76% by HPLC (area %).

Example 6: Ivermectin (2 g) with a Dᵥ(90) of 300 μm and a total impurities content of 3.22% (HPLC area %) was added to a mixture of water (26 ml), ethanol (30 ml) and formamide (3 ml) previously cooled to 0-5° C. The suspension was recirculated through a high shear mixer at 1000 RPM, for about 2 hours, while maintaining the mixture at temperature between 0° C. and 5° C. The product was filtered, and the wet product was dried at 60° C. to yield 1.77 g of ivermectin with a Dᵥ(90) of 36 μm and content of total impurities of 2.87% by HPLC (area %).

Example 7: Ivermectin (2 g) with a Dᵥ(90) of 300 μm and a total impurities content of 3.22% (HPLC area %) was added to a mixture of water (1 ml), ethanol (30 ml) and formamide (3 ml) previously cooled to 0-5° C. The suspension was recirculated through a high shear mixer at 1000 RPM, for about 2 hours, while maintaining the mixture at temperature between 0° C. and 5° C. The product was filtered, and the wet product was dried at 60° C. to yield 0.96 g of ivermectin with a Dᵥ(90) of 56 μm and content of total impurities of 2.75% by HPLC (area %).

The HPLC chromatograms were obtained with a reverse phase C18 HPLC column, dimensions 250×4 mm, pores size 5 μm, at 25° C., detection wavelength 254 nm, mobile phase water/methanol/acetonitrile (15:34:51 v/v/v), isocratic mode.

The particle size data were obtained from a Sympatec module HELOS (Helium-Neon Laser Optical System), dispersion Unit RODOS/M, potent products accessory ASPIROS, lens R5 (4.50 μm-875 μm) and R4 (1.80 μm-350 μm), conditions R5, 0.1 bar-1 bar, 18 mm/s, R4, 3 bar, 18 mm/s

### References in the FIGURES Include

Sv volume-specific surface area—total surface area of a material per bulk volume.
Sm Mass-specific surface area—total surface area of a material per unit mass.

1-26. (canceled)

27. A process for preparing ivermectin particles having reduced particle size, wherein the process comprises incorporating ivermectin particles in a liquid medium comprising a mixture of an organic solvent with water, and subjecting the medium comprising the particles to mixing in a high shear mixer.

28. A process according to claim 27 wherein the particles of ivermectin are suspended in the liquid medium.

29. A process according to claim 27 wherein the organic solvent is an alcohol, an ester, a ketone, an ether, an amide, a hydrocarbon or a halogenated hydrocarbon.

30. A process according to claim 27 wherein the organic solvent is an aliphatic alcohol, optionally wherein the aliphatic alcohol is a C₂-C₈ aliphatic alcohol.

31. A process according to claim 30 wherein the aliphatic alcohol comprises a C₂-C₄ aliphatic alcohol, optionally wherein the aliphatic alcohol is ethanol or isopropanol.

32. A process according to claim 27 wherein the medium comprising the particles further comprises an antioxidant.

33. A process according to claim 32 wherein the antioxidant is a paraben derivative, a phenol derivative or a thiol derivative.

34. A process according to claim 33 wherein the antioxidant is an alkyl paraben, optionally methyl paraben.

35. A process according to claim 33 wherein the antioxidant is an alkylated phenol derivative, optionally an alkylated phenol hydoxyanisole such as butylated hydroxyanisole; butylated hydroxy toluene or tocopherol.

36. A process according to claim 27 wherein the medium comprising the particles further comprises a stabilizer to minimize desolvation of the ivermectin.

37. A process according to claim 36 wherein the stabilizer comprises an aliphatic monocarboxylic acid amide.

38. A process according to claim 37 wherein the stabilizer comprises formamide.

39. A process according to claim 27 wherein the Dv (90) of the ivermectin particles after mixing is less than 60 microns.

40. A process according to claim 27 wherein the reduction in total impurities as measured by HPLC (% area) of the resulting ivermectin particles compared to the initial ivermectin particles is 10% or more.

41. A process according to claim 27 wherein the total impurities as measured by HPLC (% area) of the resulting ivermectin particles is 3.0% or less, optionally 2.8% or less, or 2.7% or less.

42. A process according to claim 27 wherein the medium comprising the particles comprises water; ethanol or isopropanol as the organic solvent; methylparaben as antioxidant, and formamide as a stabilizer.

43. A process according to claim 27 wherein in the mixing step a suspension of ivermectin is recirculated at a range of rotations per minute of the high shear mixer of from 500 RPM to 9000 RPM.

44. A process according to claim 43 wherein a suspension of ivermectin is recirculated at a range of rotations per minute of the high shear mixer of from 1000 RPM to 2000 RPM.

45. A process according to claim 27 wherein in the mixing step the medium comprising the ivermectin particles is recirculated at a temperature of from 0° C. to 25° C.

46. A process according to claim 45 wherein a suspension of ivermectin is recirculated at a temperature of from 0° C. and 5° C.

47. A process according to claim 27 further comprising, after the mixing, the step of washing recovered ivermectin particles with antioxidant.

48. A process according to claim 47 wherein the antioxidant is an alkyl paraben, optionally methyl paraben; or an alkylated phenol derivative, optionally an alkylated phenol hydoxyanisole such as butylated hydroxyanisole.

49. A process according to claim 47 wherein the washing step is performed at a temperature of from 0° C. to 25° C., optionally at a temperature of from 0° C. and 5° C.

50. Ivermectin particles obtainable by, or obtained by, a process according to claim 27.

51. A method comprising utilizing high shear mixing to reduce the particle size of ivermectin in a suspension of ivermectin particles in a liquid medium.

52. The method according to claim 51 wherein the liquid medium comprises water, an organic solvent comprising ethanol or isopropanol, and a stabilizer comprising formamide.
